(19) United States
(12) Patent Application Publication
(10) Pub. No.: US 2025/0269170 A1
(43) Pub. Date: Aug. 28, 2025

(54) Apparatus and Method for Treating a Condition in a Body

(71) Applicant: James M. Rynerson, Franklin, TN (US)

(72) Inventor: James M. Rynerson, Franklin, TN (US)

(21) Appl. No.: 19/199,694

(22) Filed: May 6, 2025

Related U.S. Application Data
(63) Continuation-in-part of application No. 19/059,477, filed on Feb. 21, 2025.
(60) Provisional application No. 63/557,160, filed on Feb. 23, 2024.
(60) Provisional application No. 63/643,486, filed on May 7, 2024.

(51) Int. Cl. A61N 1/05; A61N 1/36

(52) U.S. Cl. CPC A61N 1/05; A61N 1/36

(57) ABSTRACT

A method for treating a condition in a body where a biofilm on or proximate a tissue causes pain, irritation, and/or inflammation. One condition may be interstitial cystitis. The method for treating interstitial cystitis includes providing a catheter with a lumen and an elongate rod disposed within the lumen and routing the lumen through a urethra such that a distal end of the elongate rod is disposed in an interior of a bladder. Then connecting the elongate rod to a controller adapted to generate current and supplying a current at a first amperage from the controller to the distal end of the elongate rod. Applying the current for a predetermined time so as to disrupt a biofilm on or proximate to an interior wall of the bladder. A method for treating prostatitis is also disclosed.

### CROSS REFERENCE TO RELATED APPLICATION

This application is a continuation-in-part of U.S. patent application Ser. No. 19/059,477, filed Feb. 21, 2025, which claims the benefit of U.S. Provisional Patent Application Ser. No. 63/557,160, filed Feb. 23, 2024, the disclosures of which are incorporated by reference herein in its entirety. This application further claims the benefit of U.S. Provisional Patent Application Ser. No. 63/643,486, filed May 7, 2024, the disclosure of which is incorporated by reference herein in its entirety.

### TECHNICAL FIELD

The invention relates generally to an apparatus and method for treating a condition in a body.

### BACKGROUND

Interstitial cystitis (IC) is an inflamed or irritated bladder wall. It may also be referred to as painful bladder syndrome or frequency-urgency-dysuria syndrome. IC may lead to scarring and stiffening of the bladder. As such, the bladder may not be capable of holding as much urine as it did in the past. IC is a chronic disorder and the cause of IC is unknown. Symptoms of IC may include frequent urination, urgency with urination, pain and tenderness around the bladder and pelvis, and pain during sex.

Currently, there is no cure for IC. In addition, IC may be difficult to treat. The various treatments focus primarily on easing the symptoms of IC. For example, treatments may include bladder enlargement, bladder wash, medicine, transcutaneous electrical nerve stimulation (TENS), bladder training, and surgery.

The prostate gland is located just below the bladder in men. The walnut-sized prostate surrounds the top portion of the urethra that drains urine from the bladder. Prostatitis refers to a condition when the prostate gland is Inflamed. There are generally four types of prostatitis. Prostatitis may cause painful or difficult urination as well as pain in the groin, pelvic area, or genitals. Depending upon the type of prostatitis, antibiotics or pain relievers, such as alpha-blockers, may be prescribed. Neither treatment, however, may offer a lasting cure for prostatitis.

What is needed, however, is a method to effectively treat and resolve an inflamed or irritated condition in the body, such as IC and prostatitis instead of merely focusing on treating the symptoms of IC and prostatitis, for example.

### SUMMARY OF THE INVENTION

In one aspect of the disclosure, a method for treating interstitial cystitis of a patient includes generating a current at a first amperage in an interior of a bladder and applying that current for a predetermined time so as to disrupt a biofilm on or proximate to an interior wall of the bladder. In one embodiment, the predetermined time may be 1-6 minutes or 2-5 minutes.

In one embodiment, the method may further include after generating the current at the first amperage, increasing the current to a second amperage.

In one embodiment, the method may further include placing a first end of an elongate rod inside an interior of the bladder and emitting the current from the first end of the elongate rod. The method may further include operatively connecting the first end of the metal probe to a controller and changing the current from the first amperage via the controller.

In one embodiment, the elongate rod may be disposed within a lumen routed through the urethra and into the bladder.

In one embodiment, during the treatment method, the patient may be in a supine position, in a prone position, or standing during the treatment method.

In another aspect of the disclosure, a method for treating interstitial cystitis of a patient includes providing a catheter with a lumen and an elongate rod disposed within the lumen and routing the lumen through a urethra such that a distal end of the elongate rod is disposed in an interior of a bladder. The elongate rod is then connected to a controller adapted to generate current. A current is then supplied at a first amperage from the controller to the distal end of the elongate rod. The current is applied for a predetermined time so as to disrupt a biofilm on or proximate to an interior wall of the bladder. In one embodiment, the predetermined time may be 1-10 minutes or 2-5 minutes.

In one embodiment, the method may further include after generating the current at the first amperage, increasing the current to a second amperage.

In one embodiment, during the treatment method, the patient may be in a supine position, in a prone position, or standing during the treatment method.

In another aspect of the disclosure, a method for treating prostatitis of a patient includes generating a current at a first amperage in an interior of a prostate gland and applying that current for a predetermined time so as to disrupt a biofilm on or proximate to a tissue on an interior of the prostate gland. In one embodiment, the predetermined time is 1-10 minutes or 2-5 minutes.

In one embodiment, the method may further include after generating the current at the first amperage, increasing the current to a second amperage.

In another aspect of the disclosure, a method for treating prostatitis of a patient includes providing a catheter with a lumen and an elongate rod disposed within the lumen and routing the lumen through a urethra such that a distal end of the elongate rod is disposed proximate to the prostate gland. The elongate rod is then connected to a controller adapted to generate current. A current is then supplied at a first amperage from the controller to the distal end of the elongate rod. The current is applied for a predetermined time so as to disrupt a biofilm on or proximate to a tissue on an interior of the prostate gland. In one embodiment, the predetermined time may be 1-10 minutes or 2-5 minutes.

In one embodiment, the method may further include after generating the current at the first amperage, increasing the current to a second amperage.

In another aspect of the disclosure, a method for treating a condition in a body of a patient includes providing a catheter with a lumen and an elongate rod disposed within the lumen and routing the lumen into an interior of a body of a patient such that a distal end of the elongate rod is disposed proximate to a tissue in the interior of the body, where a biofilm is on or proximate to the tissue. The elongate rod is then connected to a controller adapted to generate current. A current is then supplied at a first amperage from the controller to the distal end of the elongate rod. The current is applied for a predetermined time so as to disrupt a biofilm on or proximate to the tissue. In one embodiment, the predetermined time is 1-10 minutes or 2-5 minutes. In one embodiment, the condition is one of sinusitis, gingivitis, and otitis media.

### BRIEF DESCRIPTION OF THE DRAWINGS

The accompanying drawings, which are incorporated in and constitute a part of this specification, illustrate one or more embodiments of the invention and, together with a general description of the invention given above, and the detailed description given below, serve to explain the invention.

FIG. 1A is a disassembled view of a treatment device including a flexible rod and a urinary catheter according to an embodiment of the invention.

FIG. 1B is the assembled treatment device of FIG. 1A.

FIG. 2 shows the treatment device positioned to treat a condition within the bladder.

FIG. 3 is an up-close view of the treatment device functioning to treat a condition within the bladder.

FIG. 4 is a perspective view of a controller adapted to control the treatment device during a treatment.

FIG. 5 is a close-up, cross-sectional view of the treatment device inside the bladder with the end of the flexible rob located external to the lumen of the urinary catheter according to an embodiment of the disclosure.

FIG. 6 is a close-up, cross-sectional view of the treatment device inside the bladder with the end of the flexible rob located internal to the lumen of the urinary catheter according to another embodiment of the disclosure.

FIG. 7A is a cross-section view of a treatment device according to an embodiment of the disclosure positioned in the urethra near the bladder where lumen has a side port.

FIG. 7B is a cross-sectional view of the treatment device of FIG. 7A with its end positioned inside the bladder and contacting urine therein.

FIG. 8 shows the treatment device positioned to treat a condition within the prostate gland.

FIG. 9 is an up-close view of the treatment device functioning to treat a condition within the prostate gland.

### DETAILED DESCRIPTION OF THE INVENTION

Bacteria growing on any surface of the interior of the body, including in organs, that have access to moisture will invariably grow polysaccharide/protein biofilms. If the particular strain of bacteria happens to be aggressive biofilm formers, there would be a good chance of biofilm formation on the surface. The biofilm is not an infection, but is instead a colonization that allows bacteria to produce exotoxins that lead to chronic pain and inflammation. The biofilm growing on the surface may release toxins to break down human tissue which serves as a food source for the bacteria. That release of toxins and break down of human tissue is believed to cause inflammation, including in organs.

In one example, one theory for what causes IC involves biofilm that this growing on the inside wall of the bladder. The biofilm on the inside wall of the bladder produces exotoxins that create inflammation, which is known as IC.

In another example, one theory for what causes prostatitis is biofilm growing within the prostate gland, including on any luminal surface within the prostate gland. The biofilm releases toxins and the break down of human tissue is believed to cause inflammation of the prostate gland, which is known as prostatitis.

Passing a low voltage (AC or DC) current through a biofilm will result in a devastating vaporization of that biofilm within just a few seconds to minutes. Vaporizing biofilms may be used to treat various conditions in the body, such as IC and prostatitis, for example. Other examples of conditions that may be treated by vaporizing biofilms, include, but are not limited to, sinusitis, gingivitis, and otitis media.

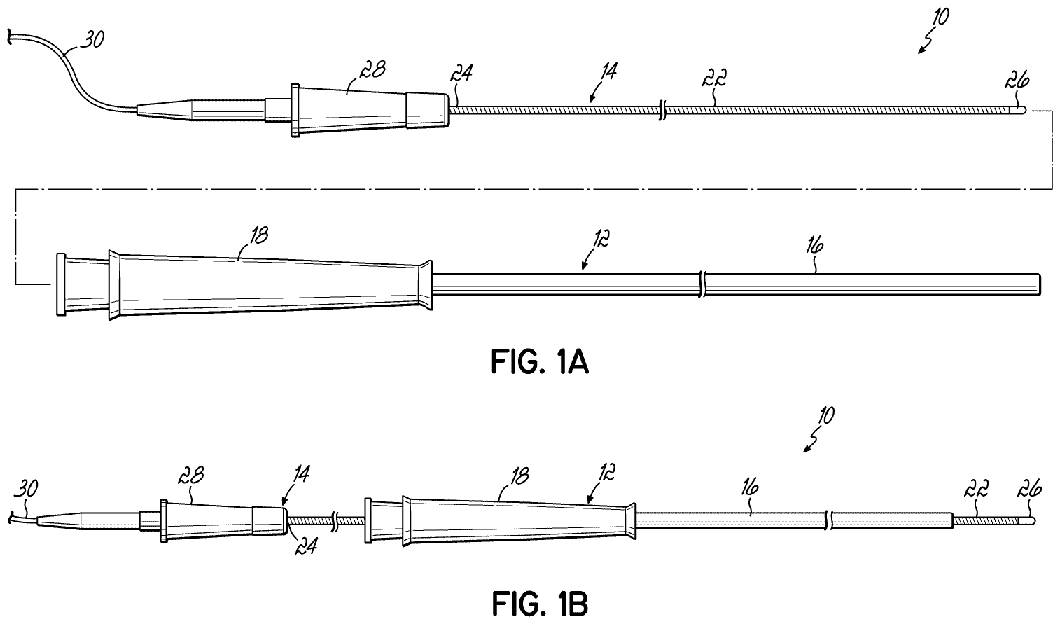

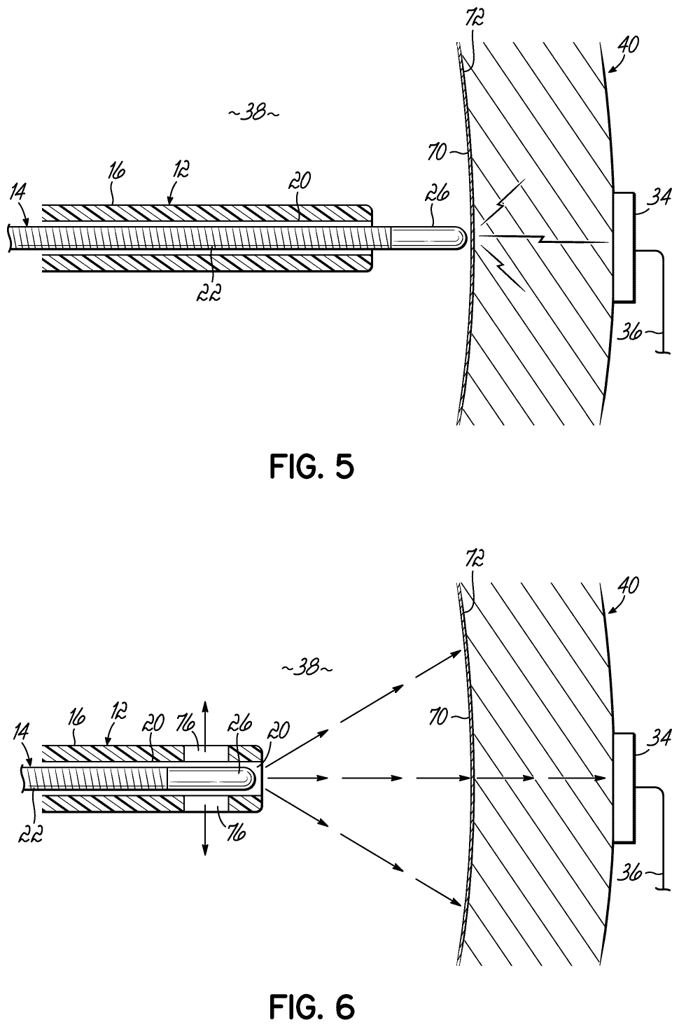

With reference to FIGS. 1A and 1B, a treatment device 10 is shown according to an embodiment of the disclosure. The treatment device 10 includes a catheter 12, such as a urinary catheter, for example, and an energy delivery device 14. The catheter 12 shown in FIGS. 1A and 1B is an intermediate urinary catheter, but any suitable catheter may be used. The urinary catheter 12 in FIGS. 1A and 1B includes a lumen 16 and a handle 18. The lumen 16 includes a central passageway 20 (FIG. 5) extending the length of the urinary catheter 12. As is typical with urinary catheters, the lumen 16 is flexible so it may be passed through the urethra and into the bladder. The energy delivery device 14 includes an elongate rod 22 adapted to fit within the central passageway 20 of the lumen. In an embodiment, the elongate rod 22 forms a water-tight seal with the central passageway 20 of the lumen 16. In one aspect, the portion of the metal rod protruding from the catheter may be sealed within the proximal end of the catheter with a silicon sealant. The elongate rod 22 may be flexible so that the elongate rod 22 may be passed through the urethra and into the bladder. The elongate rod 22 may be made from brass, copper, or any suitable electrically-conducting material. The elongate rod 22 includes a proximal end 24 and a distal end 26. The proximal end 24 is operatively coupled to a handle 28. A conductive wire 30 is operatively coupled to the proximal end 24 of the elongate rod 22. While the conductive wire 30 is shown extending through the handle 28 to reach the proximal end 24 of the elongate rod 22, other configurations may be used to connect the conductive wire 30 to the elongate rod 22. In FIG. 1B, the elongate rod 22 is positioned within the central passageway 20 of the lumen 16.

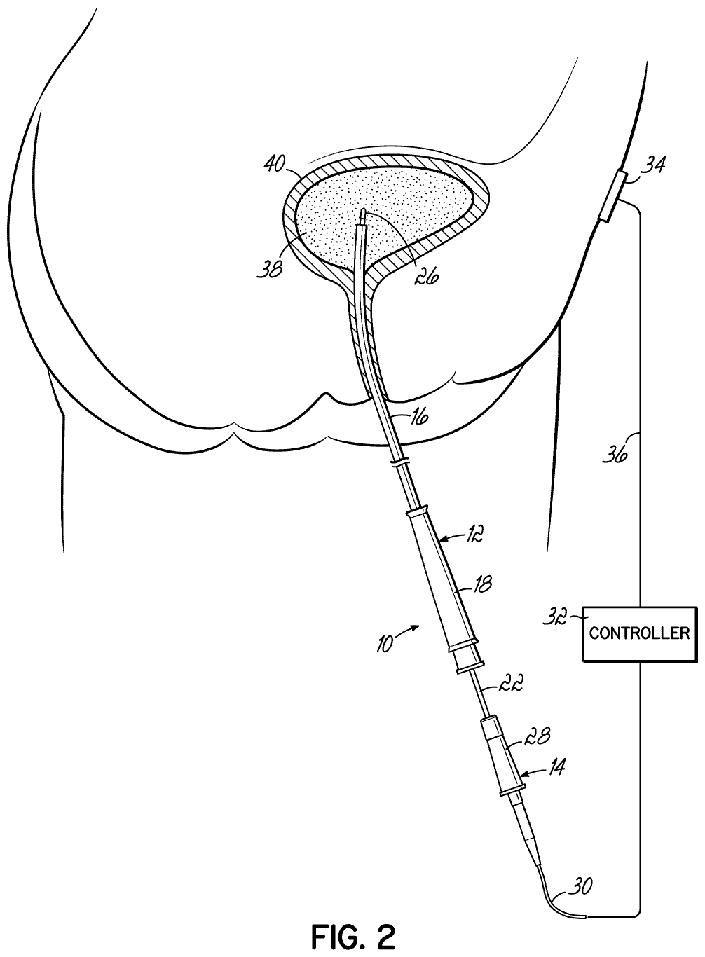

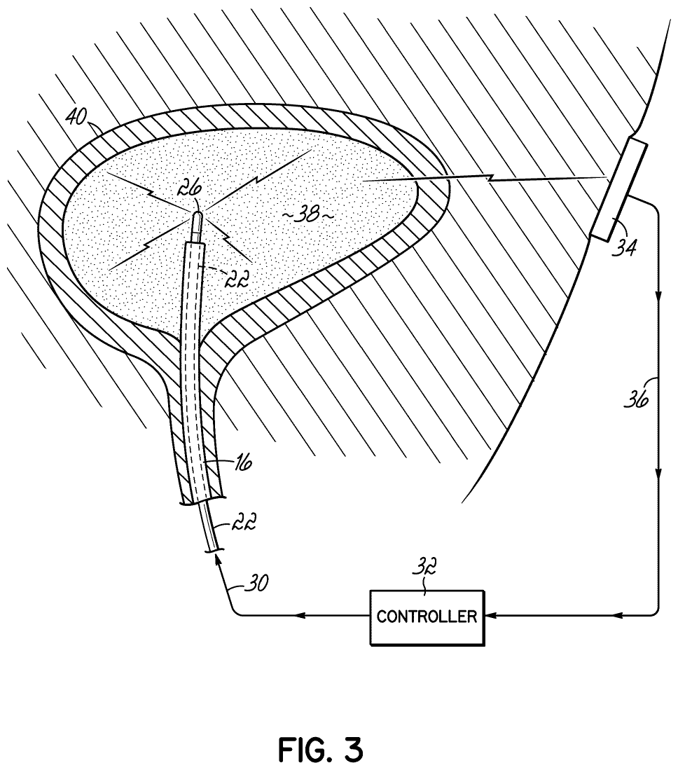

As described above, biofilm may cause inflammation in the bladder, which is known as IC. Moreover, vaporizing that biofilm may be a method for treating IC. To that end, with the elongate rod 22 positioned within the central passageway 20, the lumen 16 may then be inserted into and passed through the urethra and into the bladder as shown in FIGS. 2 and 3. The conductive wire 30 may be operatively connected to a controller 32. A grounding pad 34 may be secured to the patient's abdomen or suprapubis area. A conductive wire 36 may by operatively coupled to the grounding pad 34 and extend to and operatively couple to the controller 32.

As schematically shown in FIG. 3, the controller 32 is adapted to generate AC or DC current that passes through conductive wire 30, through elongate rod 22, through urine 38 in bladder 40, through grounding pad 34, through conductive wire 36 and back to the controller 32.

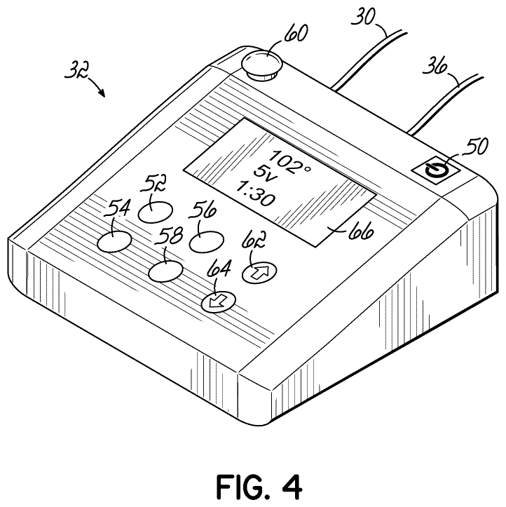

One embodiment of the controller 32 is shown in FIG. 4. The controller has an on/off button 50 and various other control buttons 52-62. Buttons 52, 54 may be used to increase or decrease the AC or DC current flowing through the elongate rod 22. Buttons 56, 58 may be used to set a timer for how long the AC or DC current will flow. The controller 32 may also include an emergency shut down button 60, which when pushed will stop the flow of AC or DC current. Buttons 62, 64 may be used to adjust other parameters during the treatment. The controller 32 may include further buttons to adjust, stop, or start a functionality of the controller 32. As used herein, the term button may include a push button, a switch, a toggle, a slide, a rotary knob, or any other device suitable for adjusting a parameter of the controller 32. The controller may further include a screen 66 which is configured to display information such as temperature of the elongate rod 22, volts, and time remaining, for example. Other information or parameters may also be shown. The temperature of the elongate rod 22 will typically increase as the AC or DC current passes through it during the treatment procedure, but the urine (or saline) in the bladder 40 will prevent any significant increase in the temperature. The temperature of the elongate rod 22 may reach a temperature of around 104° F.

FIG. 5 shows the elongate rod 22 extending from the lumen 16. In an embodiment, the distal end 26 may be rounded off smooth with approximately 2-3 mm of the distal end 26 being exposed from the end of the lumen 16. The distal end 26 of the elongate rod 22 is spaced a distance from a biofilm 70 attached to an interior wall 72 of the bladder 40. The AC or DC current is emitted from the distal end 26 and flows through the urine 38 which contains electrolytes and, therefore, is electrically conductive. Alternatively, the bladder 40 may be emptied just prior to the treatment procedure. In that circumstance, the doctor may fill the bladder 40 with saline via a separate catheter before turning on the AC or DC current in the elongate rod 22. During treatment, the AC or DC current then flows through the biofilm 70 and disrupts or vaporizes the biofilm 70. The AC or DC current then flows to the grounding pad 34 and back to the controller 32 via conductive wire 36.

FIG. 6 shows another embodiment of the disclosure where the distal end 26 of the elongate rod 22 is position within the end of the lumen 16. The end of the lumen 16 includes one or more holes 76 circumferentially arranged around the lumen 16. The one or more holes 76 permit the AC or DC current to flow out of (escape) the distal end 26 of the elongate rod 22 and into the urine filled or partially filled bladder 400.

In both FIGS. 5 and 6, the distal end 26 of the elongate rod 22 is shown relatively close to biofilm 70 and the interior wall 72 of the bladder 40. Because the urine 38 contains electrolytes and is, therefore, conductive, in general practice the distal end 26 may be positioned at or near the center of the interior of the bladder 40. The urine (or alternatively, the saline) will conduct the AC or DC current to the biofilm 70 on all or nearly all of the interior wall 72 to disrupt the biofilm 70.

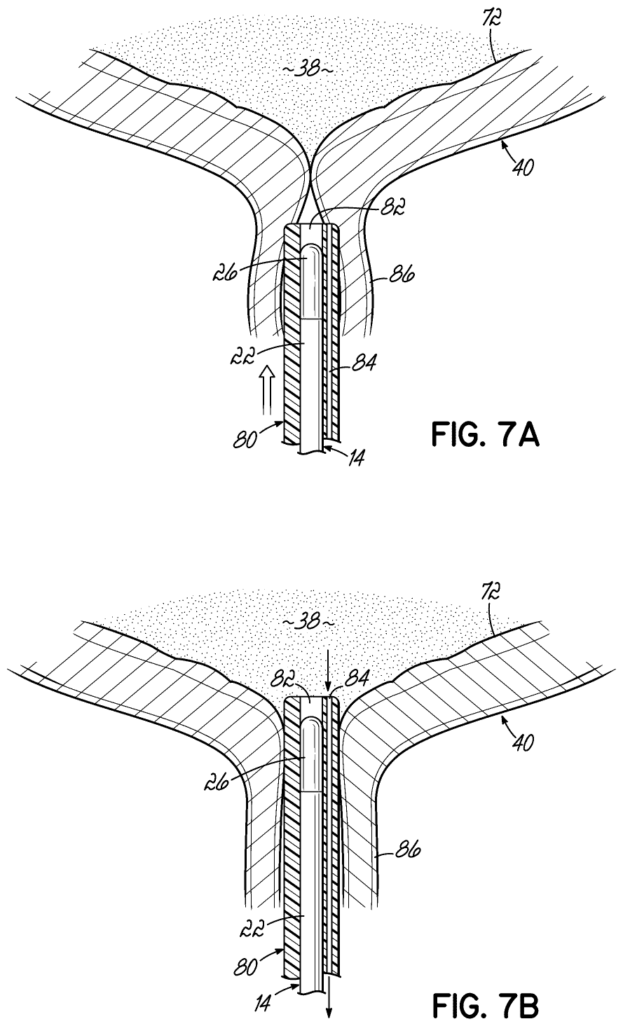

FIGS. 7A and 7B shows a lumen 80 with the elongate rod 22 inserted in a central passageway 82 of the lumen 80. The lumen 80 includes a side port 84 that extends parallel to central passageway 82. As shown in FIG. 7A, the lumen 80 is being pushed through a urethra 86, but has not yet reached the interior of the bladder 40 and the urine 38 therein. As shown in FIG. 7B, the end of the lumen 80 has entered the interior of the bladder 40 and is contacting the urine 38 therein. As explained above, the elongate rod 22 forms a water-tight seal with the central passageway 20 such that the urine 38 does not flow through the central passageway 20. The side port 84, however, adapted so that the urine 38 may flow through the side port 84 and out to the handle 18 of the lumen 16 so the doctor knows when the end of the lumen 16 has entered the bladder 40 and contacted urine 38. The doctor may then insert the lumen 16 with more precision to position it in the central portion of the interior of the bladder 40 or closer to the biofilm 70 near the interior wall 72 of the bladder 40.

In another embodiment, a very small thin strip of absorbent paper is already in place along the elongate rod 22 which will turn dark when moistened by the urine 38. The catheter 12 may also have a marking on its side to indicate the correct depth of placement of the lumen 16 into the bladder 40.

As an alternative to urinary catheter 12 with the lumen 16 and the elongate rod 22 inserted within the lumen 16, the treatment method may use an elongate rod coated with soft plastic or silicon except for an exposed distal end. Like the urinary catheter 12, the exposed distal end of the coated elongate rod conducts AC or DC current into the fluid (urine or saline) in the bladder. Current flows from the controller 32, to the elongate rod, to the urine 38 to the interior wall 72, and finally to the grounding pad 34 placed externally on the abdomen.

The invention also contemplates a method for treating a patient with interstitial cystitis. The patient is instructed to arrive for the procedure with fairly full bladder. To begin the procedure in one embodiment, the patient lies on his or her back, i.e., supine, on the exam bed. In other embodiments, the procedure may begin with the patient lying on his or her stomach, i.e., prone, or the patient standing up. The doctor inserts the lumen 16 with the elongate rod 22 in the central passageway 20 through the patient's urethra 86 and into the desired position within the patient's bladder 40. The grounding pad 34 is affixed to the skin of the patient in the desired location such as the patient's abdomen or suprapubis area. The conductive wires 30, 36 are operatively coupled to the voltage (V) output of the controller 32. Using the controller 32, the doctor increases the power (AC or DC voltage) until the patient indicates they feel something and the power is held there for approximately 1-10 minutes, and in an embodiment 2-8 minutes, and in an embodiment 2-5 minutes, although shorter or longer times may be used depending on feedback from the patient. If needed, the power may be increased to the point where it begins to feel uncomfortable. In an embodiment, the AC or DC voltage applied to the elongate rod 22 may range between 2-50 volts, but higher voltages may also be applied. As the AC or DC current flows through the urine 38 (or saline or other suitable solution) in the bladder 40, it necessarily has to pass through the biofilm 70 to reach the interior wall 72, vaporizing the biofilm 70 on the interior wall 72 of the bladder 40, which essentially eliminates the cause of the IC condition.

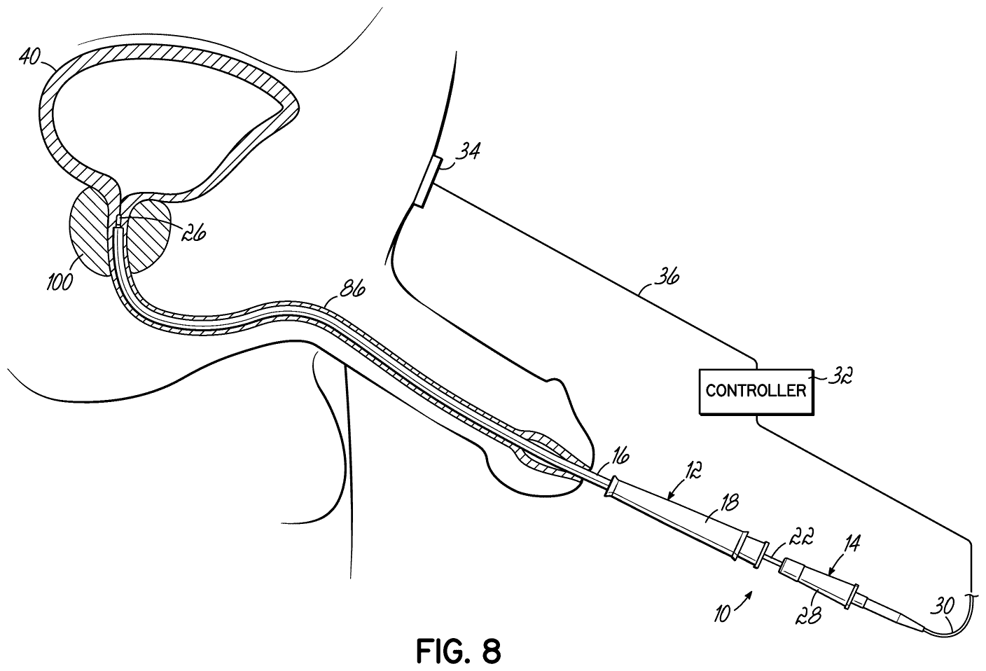

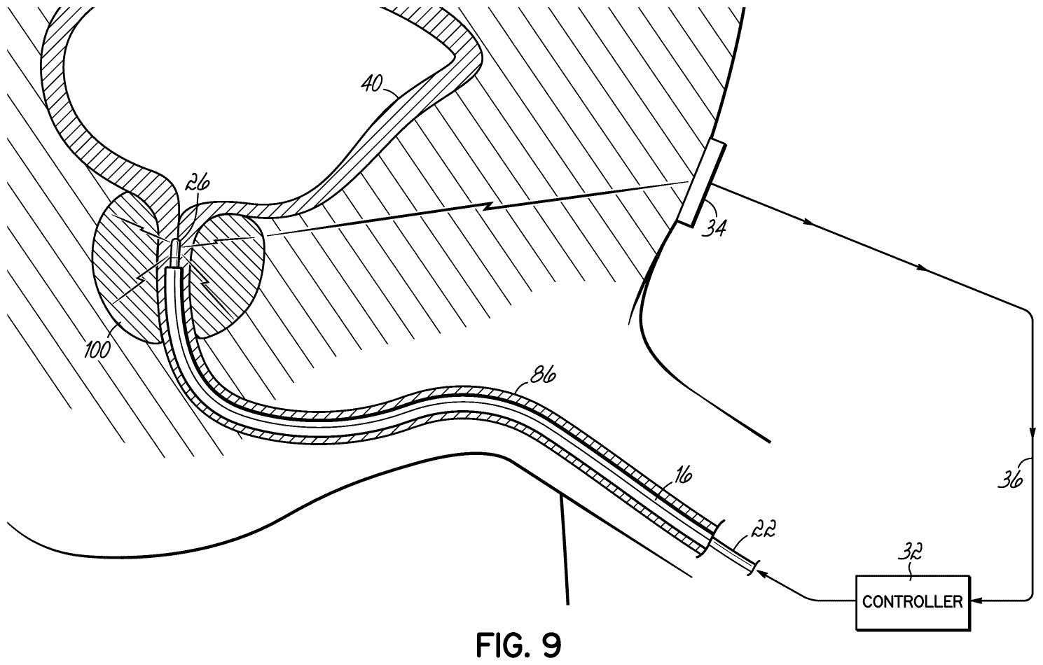

As described above, vaporizing biofilm may be one method of treating various conditions in the body. While treating IC was one example, treating prostatitis is another example. The treatment device 10 and the controller 32 may be used to treat the prostatitis. Because only males have a prostate gland 100 located just below the bladder 40 has shown in FIGS. 8 and 9, this treatment method for prostatitis is intended for use only on male patients.

To treat a male patient with prostatitis in one embodiment, the patient lies on his back, i.e., supine, on the exam bed. In other embodiments, the procedure may begin with the patient lying on his or her stomach, i.e., prone, or the patient standing up. With reference to FIGS. 8 and 9, the doctor inserts the lumen 16 with the elongate rod 22 in the central passageway 20 through the patient's urethra 86 to a point where the distal end 26 of the elongate rod 22 is positioned in the urethra 86 proximate to the prostate gland 100. The grounding pad 34 is affixed to the skin of the patient in the desired location such as the patient's abdomen or suprapubis area. The conductive wires 30, 36 are operatively coupled to the voltage (V) output of the controller 32. Using the controller 32, the doctor increases the power (AC or DC voltage) until the patient indicates they feel something and the power is held there for approximately 1-10 minutes, and in an embodiment 2-8 minutes, and in an embodiment 2-5 minutes, although shorter or longer times may be used depending on feedback from the patient. If needed, the power may be increased to the point where it begins to feel uncomfortable. In an embodiment, the AC or DC voltage applied to the elongate rod 22 may range between 2-50 volts, but higher voltages may also be applied. The AC or DC current flows through the urine 38 (or saline or other suitable solution) in the urethra 86, through urethra, and into the prostate gland 100 where the current flows through seminal and/or prostatic fluid within the prostate gland 100. As the current flows through the prostate gland 100 it passes through the biofilm 70 and vaporizing the biofilm on or proximate to a tissue on the interior of the prostate gland 100, which essentially eliminates the cause of the prostatitis. The method of treating prostatitis may be conducted with the patient is supine, prone, or standing.

Beyond specifically treating IC or prostatitis, the invention further contemplates a method for treating a condition in the body where a biofilm on or proximate a tissue is causing pain, irritation, and/or inflammation. The method for treating a condition in the body of a patient includes providing the catheter 12 with the lumen 16 and the elongate rod 22 disposed within the lumen 16. The method includes routing the lumen 16 into an interior of the body of the patient such that the distal end 26 of the elongate rod 22 is disposed proximate to a tissue in the interior of the body, where a biofilm is on or proximate to the tissue. The method further includes connecting the elongate rod 22 to the controller 32, which is adapted to generate a current. Then supplying the current at a first amperage from the controller 32 to the distal end 26 of the elongate rod 22. Finally, applying the current for a predetermined time so as to disrupt the biofilm on or proximate to the tissue. In an embodiment, the tissue may be that of an organ, such as the kidney, the lung, large intestine, the spleen, the appendix, the liver, the stomach, the pancreas, the ovaries, the thyroid, the heart, the brain, the gall bladder, the lungs, the testicles, the glands of the endocrine system, the lymphatic system, and the mammary glands. In an embodiment, the tissue may be that of the mouth (e.g., the gums), the nose/sinuses, and the ear. In an embodiment, the method may further include increasing the current to a second amperage generating the current at the first amperage. In an embodiment, the predetermined time may be 1-6 minutes or 2-5 minutes. The method of treating a condition in the body may be conducted with the patient is supine, prone, or standing.

While the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method for treating interstitial cystitis of a patient, comprising:
generating a current at a first amperage in an interior of a bladder; and
applying the current for a predetermined time so as to disrupt a biofilm on or proximate to an interior wall of the bladder.

2. The method of claim 1, wherein the predetermined time is 1-10 minutes.

3. The method of claim 2, wherein the predetermined time is 2-5 minutes.

4. The method of claim 1, further comprising:
after generating the current at the first amperage, increasing the current to a second amperage.

5. The method of claim 1, further comprising:
placing a first end of an elongate rod inside an interior of the bladder; and
emitting the current from the first end of the elongate rod.

6. The method of claim 5, further comprising:
operatively connecting the first end of the elongate rod to a controller; and
changing the current from the first amperage via the controller.

7. The method of claim 1, wherein the elongate rod is disposed within a lumen routed through a urethra and into a bladder.

8. The method of claim 1, wherein the patient is in a supine position during the treatment method.

9. The method of claim 1, wherein the patient is in a prone position during the treatment method.

10. The method of claim 1, wherein the patient is standing during the treatment method.

11. A method for treating interstitial cystitis of a patient, comprising:
providing a catheter with a lumen and an elongate rod disposed within the lumen;
routing the lumen through a urethra such that a distal end of the elongate rod is disposed in an interior of a bladder;
connecting the elongate rod to a controller adapted to generate current;
supplying a current at a first amperage from the controller to the distal end of the elongate rod; and
applying the current for a predetermined time so as to disrupt a biofilm on or proximate to an interior wall of the bladder.

12. The method of claim 11, wherein the predetermined time is 1-10 minutes.

13. The method of claim 12, wherein the predetermined time is 2-5 minutes.

14. The method of claim 11, further comprising:
after generating the current at the first amperage, increasing the current to a second amperage.

15. The method of claim 11, wherein the patient is in a supine position during the treatment method.

16. The method of claim 11, wherein the patient is in a prone position during the treatment method.

17. The method of claim 11, wherein the patient is standing during the treatment method.

18. A method for treating prostatitis of a patient, comprising:
generating a current at a first amperage in an interior of a prostate gland; and
applying the current for a predetermined time so as to disrupt a biofilm on or proximate to a tissue on an interior of the prostate gland.

19. The method of claim 18, wherein the predetermined time is 1-10 minutes.

20. The method of claim 19, wherein the predetermined time is 2-5 minutes.

21. The method of claim 18, further comprising:
after generating the current at the first amperage, increasing the current to a second amperage.

22. A method for treating prostatitis of a patient, comprising:
providing a catheter with a lumen and an elongate rod disposed within the lumen;
routing the lumen through a urethra such that a distal end of the elongate rod is disposed proximate to the prostate gland;
connecting the elongate rod to a controller adapted to generate current;
supplying a current at a first amperage from the controller to the distal end of the elongate rod; and
applying the current for a predetermined time so as to disrupt a biofilm on or proximate to a tissue on an interior of the prostate gland.

23. The method of claim 22, wherein the predetermined time is 1-10 minutes.

24. The method of claim 23, wherein the predetermined time is 2-5 minutes.

25. The method of claim 22, further comprising:
after generating the current at the first amperage, increasing the current to a second amperage.

26. A method for treating a condition in a body of a patient, comprising:
providing a catheter with a lumen and an elongate rod disposed within the lumen;
routing the lumen into an interior of a body of a patient such that a distal end of the elongate rod is disposed proximate to a tissue in the interior of the body, where a biofilm is on or proximate to the tissue;
connecting the elongate rod to a controller adapted to generate current;
supplying a current at a first amperage from the controller to the distal end of the elongate rod; and
applying the current for a predetermined time so as to disrupt a biofilm on or proximate to the tissue.

27. The method of claim 26, wherein the predetermined time is 1-10 minutes.

28. The method of claim 27, wherein the predetermined time is 2-5 minutes.

29. The method of claim 26, where the condition is one of sinusitis, gingivitis, and otitis media.
